(19) United States
(12) Patent Application Publication
(10) Pub. No.: US 2023/0133587 A1
(43) Pub. Date: May 4, 2023

(54) Amino Acid Mediated Gene Delivery and Its Uses

(71) Applicant: CHINESE INSTITUTE FOR BRAIN RESEARCH, BEIJING, Beijing (CN)

(72) Inventors: Wenzhi SUN, Beijing (CN); Chunhai FAN, Shanghai (CN); Lihua WANG, Shanghai (CN); Ji HU, Shanghai (CN); Haibin ZHOU, Beijing (CN)

(21) Appl. No.: 17/914,065

(22) PCT Filed: Mar. 26, 2020
(86) PCT No.: PCT/CN2020/081405

§ 371 Date: Sep. 23, 2022

(51) Int. Cl. C12N 15/87; C12N 15/85; C12N 15/90; C12N 15/11; C12N 9/22; A61K 47/18; A61K 31/713

(52) U.S. Cl. CPC C12N 15/87; C12N 15/85; C12N 15/907; C12N 15/11; C12N 9/22; A61K 47/183; A61K 31/713; C12N 2800/107; C12N 2310/20; C12N 2800/80

(57) ABSTRACT

Provided is an application of an amino acid as a vehicle for delivery of a nucleic acid into the nervous system. By local injection, preferably, combined with means of stereotactic injection, immunofluorescence staining, confocal microscopy, etc., the single-stranded oligonucleotides are efficiently delivered into brain cells, and the plasmid vectors are also delivered into astrocytes in mouse brain.

### CROSS-REFERENCE TO RELATED APPLICATION

This application is a U.S. national phase of International Patent Application No. PCT/CN2020/081405 filed on Mar. 26, 2020, the entirety of which is incorporated herein by reference.

### INTRODUCTION

Numerous central nervous system disorders are associated with abnormal gene expression. In the past decades, gene therapy has become a vital means for the treatment of various central nervous system diseases. However, safe and efficient strategies for gene delivery into the central nervous system are very limited. Currently, the primary approach for gene delivery in gene therapy generally uses viral vectors, which have a series of potential risks in gene therapy due to its inherent properties. Also, several antisense oligonucleotides drugs are being utilized in treatment of neurodegenerative disorders, and repeated high-dose administration for a long time is necessary to have an effective treatment. Although other non-viral vectors (such as cationic polymers, liposomes, nanocarriers, etc.) have attracted wide attention, it is difficult to achieve high efficiency and low cytotoxicity with these non-viral vectors because of blood-brain barrier and heterogeneity of brain cells. Therefore, the development of new delivery strategies, for example, new vehicles for nucleic acid materials is of great significance to the therapeutics of neurological disorders, especially to the gene therapy of neurodegenerative diseases.

Many neurological diseases are related to abnormal gene expression. Gene therapy has brought dawn to the treatment of these neurological diseases. In recent years, more and more studies have found that astrocyte dysfunction can cause a series of central nervous system disorders. As a result, the regulation of astrocyte gene expression has drew wide attention for a deeper understanding of the function of astrocytes in brain tissue and the treatment of related diseases.

Currently, the primary approach for gene delivery in the central nervous system is the use of viral vectors. However, several limitations of viral vectors have restrained their application, especially in clinics, such as the limited capcity which can only carry small piece of foreign genes, and the strong immunogenicity which may lead to severe inflammatory storm and potential carcinogenic risks. Therefore, the development of non-viral gene delivery approach has become an important research direction for gene delivery in the central nervous system. Currently used non-viral approaches generally have low delivery efficiency, high cytotoxicity, and lack cell-type-specificity. There is a need for non-viral based vehicles which can satisfy high delivery efficiency, low cytotoxicity and cell-type-specificity.

### SUMMARY OF THE INVENTION

The present invention focuses on improving the delivery efficiency of nucleic acid materials (such as oligonucleotides or plasmid vectors) into brain tissue. By local injection, preferably, combined with means of stereotactic injection, immunofluorescence staining, confocal microscopy, etc., the single-stranded oligonucleotides are efficiently delivered into brain cells, and the plasmid vectors are also delivered into astrocytes in mouse brain. And using these strategies, we have achieved knock-out and knock-in of genes in astrocytes in vivo. The present invention firstly discovers that an amino acids can be directly used as a vehicle for delivery of nucleic acid into tissues or organs of living organisms, in particular, the central nervous system.

In the first place, the present invention provides a vehicle system for delivery into living organisms, wherein the vehicle system comprises an amino acid and a nucleic acid.

The nucleic acid is selected from oligonucleotide and plasmid. The oligonucleotide may be short length single-stranded DNA, RNA, such as antisense RNA and siRNA.

Preferably, the vehicle system comprises an amino acid and one or more plasmids.

Preferably, the amino acid is Glycine, GABA (γ-aminobutyric acid), Proline, Alanine, Serine, Histidine, or Threonine. The amino acid may be L-format or D-format. Preferably, the amino acid is L-Proline, D-Alanine, L-Serine, Glycine, GABA (γ-aminobutyric acid), L-Histidine, or L-Threonine.

The nucleic acid(s) is dissolved in the solution comprising an amino acid before delivery. Alternatively, the nucleic acid(s) and the amino acid solution are delivered separately.

The vehicle system of the present invention can be directly delivered into tissues or organs of living organisms. Living organisms include but are not limited to plant and animals. The animals are invertebrates or vertebrates. Preferably, the animals are birds, chicken, ducks, geese. Preferably, the animals are mammals, such as mice, rats, cats, rabbits, canines, horses, cows, sheep, goats, pigs, tree shrews, monkeys, chimpanzees, human beings, etc.

Preferably, the tissue or organ is the central nervous system, including the brain and spinal cord.

The brain involves but is not limited to midbrain, thalamus, hypothalamus, brainstem, cerebellum, globus pallidus lateralis, cerebral cortex, and hippocampus.

Preferably, the vehicle system of the present invention can be directly delivered into astrocytes within midbrain, thalamus, hypothalamus, brainstem, cerebellum, globus pallidus lateralis, cerebral cortex and hippocampus via the vehicle system of the present invention.

Preferably, the vehicle system of the present invention can be directly into Bergman glial cells, velate astrocytes, and basket/stellate intermediate neuron within the cerebellum via the vehicle system of the present invention.

Preferably, the uptake of plasmid by astrocytes via the vehicle system of the present invention is of concentration-dependence. The concentration of the amino acid in the solution of the vehicle system can be up to 300 mM. Preferably, the concentration of the amino acid in the solution of the vehicle system is in the range of 50 mM-300 mM.

Preferably, Glycine specifically promotes astrocyte uptake of plasmid vectors. Preferably, GABA specifically promotes astrocyte uptake of plasmid vectors.

The osmotic pressure of glycine solution at a concentration of 300 mM is roughly equivalent to that of physiological saline, and the osmotic pressure of the solution is proportional to the concentration. In the present invention, it is determined that the pH value of the glycine solution at different concentrations remains stable and maintains at pH=6.

Similarly, the solutions of L-Proline, D-Alanine, L-Serine, L-Histidine, and L-Threonine all promote astrocyte uptake of plasmid vector(s).

Preferably, the vehicle system of the present invention further comprises a selective inhibitor of amino acid transporter in addition to an amino acid. For example, the efficiency of uptake of plasmid by astrocytes via the vehicle system comprising glycine and bitopertin (a selective inhibitor of Glycine transporter 1) is 3-4 times higher than the vehicle system comprising glycine.

In another embodiment, the vehicle system of the present invention efficiently delivers an oversized plasmid vector carrying the CRISPR/Cas9 system to astrocytes and successfully knocks out a gene of a transgenic mouse genome.

In another embodiment, the vehicle system of the present invention also successfully knocks the exogenous gene into the astrocyte genome to achieve permanent expression of the exogenous gene.

At the same time, the piggyBac transposon system was used to successfully knock the exogenous gene eGFP into the astrocyte genome to achieve permanent expression of the exogenous gene.

The vehicle system of the present invention can promote efficient co-transfection of multiple plasmid vectors without using specific promoters, viral vector, and transgenic mice. Preferably, the vehicle system of the present invention can achieve efficient cotransfection of two or three plasmid vectors into astrocytes.

The vehicle system of the present invention can be used in gene therapy for treating diseases of the nervous system, especially disorders of the central nervous system. For example, the diseases may be neurodegenerative diseases, including Alzheimer's disease (AD), Parkinson's disease (PD), Huntington's disease (HD), amyotrophic lateral sclerosis (ALS), and spinal cerebellar ataxia. The diseases may be neurogenetic diseases, for example, congenital spinal muscular atrophy. The diseases may be tumors in the brain and/or spinal cord, for example, glioma.

The efficient co-transfection of multiple plasmid vectors, provides a powerful tool for manipulating the expression of multiple genes in astrocytes simultaneously.

In the second place, the present invention provides the use of an amino acid as a vehicle for the delivery of nucleic acid into living organisms.

The nucleic acid is selected from oligonucleotide and plasmid. The oligonucleotide may be short length single-stranded DNA, RNA, such as antisense RNA and siRNA.

Preferably, the vehicle is a solution comprising an amino acid.

Preferably, the vehicle of the present invention can deliver one or more plasmids simultaneously.

Preferably, the amino acid is Glycine, GABA (γ-Aminobutyric acid), Proline, Alanine, Serine, Histidine, or Threonine. The amino acid may be L-format or D-format. Preferably, the amino acid is L-Proline, D-Alanine, L-Serine, Glycine, GABA (γ-Aminobutyric acid), L-Histidine, or L-Threonine.

The nucleic acid(s) is dissolved in the solution comprising an amino acid before delivery. Alternatively, the nucleic acid(s) and the amino acid solution are delivered separately.

The nucleic acid can be directly delivered into tissues or organs of living organisms. Living organisms include but are not limited to plant and animals. The animals are invertebrates or vertebrates. Preferably, the animals are birds, chicken, ducks, geese. Preferably, the animals are mammals, such as mice, rats, cats, rabbits, canines, horses, cows, sheep, goats, pigs, tree shrews, monkeys, chimpanzees, human beings, etc.

Preferably, the tissue or organ is the central nervous system, including the brain and spinal cord.

The brain involves but is not limited to midbrain, thalamus, hypothalamus, brainstem, cerebellum, globus pallidus lateralis, cerebral cortex, and hippocampus.

Preferably, the nucleic acid can be directly delivered into astrocytes within midbrain, thalamus, hypothalamus, brainstem, cerebellum, globus pallidus lateralis, cerebral cortex, and hippocampus via the vehicle of the present invention.

Preferably, the nucleic acid can be directly delivered into Bergman glial cells, velate astrocytes, and basket/stellate intermediate neuron within cerebellum via the vehicle of the present invention.

Preferably, the uptake of plasmid by astrocytes via the vehicle of the present invention is of concentration-dependence. The concentration of the amino acid can be up to 300 mM. Preferably, the concentration of the amino acid is in the range of 50 mM-300 mM.

Preferably, Glycine specifically promotes astrocyte uptake of plasmid vectors. Preferably, GABA specifically promotes astrocyte uptake of plasmid vectors.

The osmotic pressure of glycine solution at a concentration of 300 mM is roughly equivalent to that of physiological saline, and the osmotic pressure of the solution is proportional to the concentration. In the present invention, it is determined that the pH value of the glycine solution at different concentrations remains stable and maintains at pH=6.

Similarly, the solutions of L-Proline, D-Alanine, L-Serine, L-Histidine, and L-Threonine all promote astrocyte uptake of plasmid vector(s).

Preferably, the vehicle of the present invention further comprises a selective inhibitor of amino acid transporter. For example, the efficiency of uptake of plasmid by astrocytes via the vehicle comprising glycine and bitopertin (a selective inhibitor of Glycine transporter 1) is 3-4 times higher than the vehicle merely containing glycine.

In another embodiment, the vehicle of the present invention efficiently delivers an oversized plasmid vector carrying the CRISPR/Cas9 system to astrocytes and successfully knocks out a gene of a transgenic mouse genome.

The vehicle of the present invention also successfully knocks the exogenous gene into the astrocyte genome to achieve permanent expression of the exogenous gene.

The vehicle of the present invention can promote efficient co-transfection of multiple plasmid vectors without using specific promotor, viral vector, and transgenic mice. Preferably, the vehicle system of the present invention can achieve efficient cotransfection of two or three plasmid vectors into astrocytes.

The efficient co-transfection of multiple plasmid vectors provides a powerful tool for manipulating the expression of multiple genes in astrocytes simultaneously.

The vehicle of the present invention can be used in gene therapy for treating diseases of the nervous system, especially disorders of central nervous system. For example, the diseases may be neurodegenerative diseases, including Alzheimer's disease (AD), Parkinson's disease (PD), Huntington's disease (HD), amyotrophic lateral sclerosis (ALS), and spinal cerebellar ataxia. The diseases may be neurogenetic diseases, for example, congenital spinal muscular atrophy. The diseases may be tumors in the brain and/or spinal cord, in particular, for example, glioma.

The efficient co-transfection of multiple plasmid vectors, provides a powerful tool for manipulating the expression of multiple genes in astrocytes simultaneously.

In the third place, the present invention provides a method for delivery of nucleic acid into living organisms through using an amino acid as a vehicle.

The nucleic acid is selected from oligonucleotide and plasmid. The oligonucleotide may be short length single-stranded DNA, RNA, such as antisense RNA and siRNA.

Preferably, the vehicle is a solution comprising an amino acid.

Preferably, the vehicle of the present invention can deliver one or more plasmids simultaneously.

The nucleic acid is dissolved in the solution comprising an amino acid before delivery. Alternatively, the nucleic acid and the amino acid solution are delivered separately.

Preferably, the amino acid is Glycine, GABA (γ-aminobutyric acid), Proline, Alanine, Serine, Histidine, or Threonine. The amino acid may be L-format or D-format. Preferably, the amino acid is L-Proline, D-Alanine, L-Serine, Glycine, GABA (γ-aminobutyric acid), L-Histidine, or L-Threonine.

The nucleic acid can be directly delivered into tissues or organs of living organisms. Living organisms include but are not limited to plant and animals. The animals are invertebrates or vertebrates. Preferably, the animals are birds, chicken, ducks, geese. Preferably, the animals are mammals, such as mice, rats, cats, rabbits, canines, horses, cows, sheep, goats, pigs, tree shrews, monkeys, chimpanzees, human beings, etc.

Preferably, the tissue or organ is the central nervous system, including the brain and spinal cord.

The brain involves but is not limited to midbrain, thalamus, hypothalamus, brainstem, cerebellum, globus pallidus lateralis, cerebral cortex, and hippocampus.

Preferably, the nucleic acid can be directly delivered into astrocytes within midbrain, thalamus, hypothalamus, brainstem, cerebellum, globus pallidus lateralis, cerebral cortex, and hippocampus via the vehicle of the present invention.

Preferably, the nucleic acid can be directly delivered into Bergman glial cells, velate astrocytes, and basket/stellate intermediate neuron within the cerebellum via the vehicle of the present invention.

Preferably, the uptake of plasmid by astrocytes via the vehicle of the present invention is of concentration-dependence. The concentration of the amino acid can be up to 300 mM. Preferably, the concentration of the amino acid is in the range of 50 mM-300 mM.

Preferably, Glycine specifically promotes astrocyte uptake of plasmid vectors. Preferably, GABA specifically promotes astrocyte uptake of plasmid vectors.

The osmotic pressure of glycine solution at a concentration of 300 mM is roughly equivalent to that of physiological saline, and the osmotic pressure of the solution is proportional to the concentration. In the present invention, it is determined that the pH value of the glycine solution at different concentrations remains stable and maintains at pH=6.

Similarly, the solutions of L-Proline, D-Alanine, L-Serine, L-Histidine, and L-Threonine all promote astrocyte uptake of plasmid vector(s).

Preferably, the vehicle of the present invention further comprises a selective inhibitor of amino acid transporter. For example, the efficiency of uptake of plasmid by astrocytes via the vehicle comprising glycine and bitopertin (a selective inhibitor of Glycine transporter 1) is 3-4 times higher than the vehicle merely containing glycine.

The vehicle of the present invention efficiently delivers an oversized plasmid vector carrying the CRISPR/Cas9 system to astrocytes and successfully knocks out a gene of a transgenic mouse genome.

The vehicle of the present invention also successfully knocks the exogenous gene into the astrocyte genome to achieve permanent expression of the exogenous gene.

The vehicle of the present invention can promote efficient co-transfection of multiple plasmid vectors without using specific promotor, viral vector, and transgenic mice. Preferably, the vehicle system of the present invention can achieve efficient cotransfection of two or three plasmid vectors into astrocytes.

The efficient co-transfection of multiple plasmid vectors, provides a powerful tool for manipulating the expression of multiple genes in astrocytes simultaneously.

In the fourth place, the present invention provides the use of the vehicle system in the first place for delivery of nucleic acid into living organisms.

The nucleic acid is selected from oligonucleotide and plasmid. The oligonucleotide may be short length single-stranded DNA, RNA, such as antisense RNA and siRNA.

Preferably, the vehicle system comprises an amino acid and one or more plasmids.

The nucleic acid is dissolved in the solution comprising an amino acid before delivery. Alternatively, the nucleic acid and the amino acid solution are delivered separately.

Preferably, the amino acid is Glycine, GABA (γ-Aminobutyric acid), Proline, Alanine, Serine, Histidine, or Threonine. The amino acid may be L-format or D-format. Preferably, the amino acid is L-Proline, D-Alanine, L-Serine, Glycine, GABA (γ-Aminobutyric acid), L-Histidine, or L-Threonine.

The vehicle system of the present invention can be directly delivered into tissues or organs of living organisms. Living organisms include but are not limited to plant and animals. The animals are invertebrates or vertebrates. Preferably, the animals are birds, chicken, ducks, geese. Preferably, the animals are mammals, such as mice, rats, cats, rabbits, canines, horses, cows, sheep, goats, pigs, tree shrews, monkeys, chimpanzees, human beings, etc.

Preferably, the tissue or organ is the central nervous system, including the brain and spinal cord.

The brain involves but is not limited to midbrain, thalamus, hypothalamus, brainstem, cerebellum, globus pallidus lateralis, cerebral cortex, and hippocampus.

Preferably, the vehicle system of the present invention can be directly delivered into astrocytes within midbrain, thalamus, hypothalamus, brainstem, cerebellum, globus pallidus lateralis, cerebral cortex and hippocampus via the vehicle system of the present invention.

Preferably, the vehicle system of the present invention can be directly into Bergman glial cells, velate astrocytes, and basket/stellate intermediate neuron within the cerebellum via the vehicle system of the present invention.

Preferably, the uptake of plasmid by astrocytes via the vehicle system of the present invention is of concentration-dependence. The concentration of the amino acid in the solution of vehicle system can be up to 300 mM. Preferably, the concentration of the amino acid in the solution of vehicle system is in the range of 50 mM-300 mM.

Preferably, Glycine specifically promotes astrocyte uptake of plasmid vectors. Preferably, GABA specifically promotes astrocyte uptake of plasmid vectors.

Similarly, the solutions of L-Proline, D-Alanine, L-Serine, L-Histidine, and L-Threonine all promote astrocyte uptake of plasmid vector(s).

Preferably, the vehicle system of the present invention further comprises a selective inhibitor of amino acid transporter in addition to an amino acid. For example, the efficiency of uptake of plasmid by astrocytes via the vehicle system comprising glycine and bitopertin (a selective inhibitor of Glycine transporter 1) is 3-4 times higher than the vehicle system comprising glycine.

The vehicle system of the present invention efficiently delivers an oversized plasmid vector carrying the CRISPR/Cas9 system to astrocytes, and successfully knocks out a gene of a transgenic mouse genome.

The vehicle system of the present invention also successfully knocks the exogenous gene into the astrocyte genome to achieve permanent expression of the exogenous gene.

The vehicle system of the present invention can promote efficient co-transfection of multiple plasmid vectors without using specific promotor, viral vector, and transgenic mice. Preferably, the vehicle system of the present invention can achieve efficient cotransfection of two or three plasmid vectors into astrocytes.

The efficient co-transfection of multiple plasmid vectors provides a powerful tool for manipulating the expression of multiple genes in astrocytes simultaneously.

The vehicle system of the present invention can be used in gene therapy for treating diseases of the nervous system, especially diseases of the central nervous system. For example, the diseases may be neurodegenerative diseases, for example epilepsy, Alzheimer's disease (AD), Parkinson's disease (PD), Huntington's disease (HD), amyotrophic lateral sclerosis (ALS), and spinal cerebellar ataxia. The diseases may be neurogenetic diseases, for example, congenital spinal muscular atrophy. The diseases may be tumors in the brain and/or spinal cord.

In the fifth place, the present invention provides the use of the vehicle system in the first place in gene therapy for treating diseases of the nervous system, especially diseases of central nervous system. For example, the diseases may be neurodegenerative diseases, for example, Alzheimer's disease (AD), Parkinson's disease (PD), Huntington's disease (HD), amyotrophic lateral sclerosis (ALS), and spinal cerebellar ataxia. The diseases may be neurogenetic diseases, for example, congenital spinal muscular atrophy. The diseases may be tumors in the brain and/or spinal cord, for example, glioma.

The vehicle system of the present invention can be directly delivered into the nervous system of animals, in particular, the central nervous system. The animals are invertebrates or vertebrates. Preferably, the animals are birds, chicken, ducks, geese. Preferably, the animals are mammals, such as mice, rats, cats, rabbits, canines, horses, cows, sheep, goats, pigs, tree shrews, monkeys, chimpanzees, human beings, etc.

Preferably, the nervous system is the central nervous system, including the brain and spinal cord.

The brain involves but is not limited to midbrain, thalamus, hypothalamus, brainstem, cerebellum, globus pallidus lateralis, cerebral cortex, and hippocampus.

Preferably, the vehicle system of the present invention can be directly delivered into astrocytes within midbrain, thalamus, hypothalamus, brainstem, cerebellum, globus pallidus lateralis, cerebral cortex and hippocampus via the vehicle system of the present invention.

Preferably, the vehicle system of the present invention can be directly into Bergman glial cells, velate astrocytes, and basket/stellate intermediate neuron within cerebellum via the vehicle system of the present invention.

Preferably, the uptake of plasmid by astrocytes via the vehicle system of the present invention is of concentration-dependence. The concentration of the amino acid in the solution of vehicle system can be up to 300 mM. Preferably, the concentration of the amino acid in the solution of vehicle system is in the range of 50 mM-300 mM.

Preferably, Glycine specifically promotes astrocyte uptake of plasmid vectors. Preferably, GABA specifically promotes astrocyte uptake of plasmid vectors.

The osmotic pressure of glycine solution at a concentration of 300 mM is roughly equivalent to that of physiological saline, and the osmotic pressure of the solution is proportional to the concentration. In the present invention, it is determined that the pH value of the glycine solution at different concentrations remains stable and maintains at pH=6.

Similarly, the solutions of L-Proline, D-Alanine, L-Serine, L-Histidine, and L-Threonine all promote astrocyte uptake of plasmid vector(s).

Preferably, the vehicle system of the present invention further comprises a selective inhibitor of amino acid transporter in addition to an amino acid. For example, the efficiency of uptake of plasmid by astrocytes via the vehicle system comprising glycine and bitopertin (a selective inhibitor of Glycine transporter 1) is 3-4 times higher than the vehicle system comprising glycine.

The vehicle system of the present invention efficiently delivers an oversized plasmid vector carrying the CRISPR/Cas9 system to astrocytes and successfully knocks out a gene of a transgenic mouse genome. Preferably, the vehicle system is the vehicle system comprising Glycine.

The vehicle system of the present invention also successfully knocks the exogenous gene into the astrocyte genome to achieve permanent expression of the exogenous gene.

In particular, the transposon system is delivered into astrocyte, and thus the permanent expression of foreign genes in adult mouse astrocytes was achieved.

The vehicle system of the present invention can promote efficient co-transfection of multiple plasmid vectors without using specific promotor, viral vector, and transgenic mice. Preferably, the vehicle system of the present invention can achieve efficient cotransfection of two or three plasmid vectors into astrocytes.

The efficient co-transfection of multiple plasmid vectors, provides a powerful tool for manipulating the expression of multiple genes in astrocytes simultaneously.

The gene(s) to be delivered into the nervous system of animals, in particular, the central nervous system, via the vehicle system of the present invention can be selected as per the diseases to be treated.

In the sixth place, the present invention provides a vehicle system for delivery a CRISPR/Cas9 system into central nervous system so as to knock out one or more genes in an animal. In particular, the vehicle system for delivery a CRISPR/Cas9 system can be delivered into astrocytes.

The vehicle system comprises an amino acid and a CRISPR/Cas9 system.

CRISPR/Cas9 system is dissolved in the solution comprising the amino acid before delivery. Alternatively, the CRISPR/Cas9 system and the amino acid solution are delivered separately.

Preferably, the amino acid is Glycine, GABA (γ-Aminobutyric acid), Proline, Alanine, Serine, Histidine, or Threonine. The amino acid may be L-format or D-format. Preferably, the amino acid is L-Proline, D-Alanine, L-Serine, Glycine, GABA (γ-Aminobutyric acid), L-Histidine, or L-Threonine. Preferably, the amino acid is Glycine.

The animals are invertebrates or vertebrates. Preferably, the animals are birds, chicken, ducks, geese. Preferably, the animals are mammals, such as mice, rats, cats, rabbits, canines, horses, cows, sheep, goats, pigs, tree shrews, monkeys, chimpanzees, human beings, etc.

Therefore, the vehicle system for delivery a CRISPR/Cas9 system in the present invention can be used in gene therapy for tumors, neurodegenerative diseases, for example, Alzheimer's disease (AD), Parkinson's disease (PD), Huntington's disease (HD), amyotrophic lateral sclerosis (ALS), and spinal cerebellar ataxia, and neurogenetic diseases, for example, congenital spinal muscular atrophy. The tumor can be in the brain and/or spinal cord, for example, glioma.

In the seventh place, the present invention provides a vehicle system for delivery of a transposon system into astrocytes, wherein the vehicle system comprises an amino acid.

The transposon system is delivered into astrocyte, and thus the permanent expression of foreign genes in adult mouse astrocytes was achieved.

Preferably, the transposon system is the piggyBac transposon system. The Brainbow technology for long-term tracking astrocytes of adulthood mice can be constructed by the piggyBac transposon system.

Preferably, the amino acid is Glycine, GABA (γ-aminobutyric acid), Proline, Alanine, Serine, Histidine, or Threonine. The amino acid may be L-format or D-format. Preferably, the amino acid is L-Proline, D-Alanine, L-Serine, Glycine, GABA (γ-aminobutyric acid), L-Histidine, or L-Threonine.

The transposon is dissolved in the solution comprising an amino acid before delivery. Alternatively, the transposon and the amino acid solution are delivered separately.

The concentration of the amino acid in the solution of vehicle system can be up to 300 mM. Preferably, the concentration of the amino acid in the solution of vehicle system is in the range of 50 mM-300 mM.

In the eighth place, the present invention provides a vehicle system for delivery one or more kinds of vectors expressing one or more fluorescent proteins into astrocytes.

The vehicle system is a solution comprising an amino acid and one or more kinds of vectors expressing one or more fluorescent proteins. Preferably, the vectors are plasmids.

Preferably, the amino acid is Glycine, GABA (γ-Aminobutyric acid), Proline, Alanine, Serine, Histidine, or Threonine. The amino acid may be L-format or D-format. Preferably, the amino acid is L-Proline, D-Alanine, L-Serine, Glycine, GABA (γ-Aminobutyric acid), L-Histidine, or L-Threonine. Preferably, the amino acid is Glycine.

Preferably, the vehicle system comprises two or three plasmids. The fluorescent proteins may be RFP (red), YFP or GFP (green), or CFP or BFP (blue).

The present invention successfully constructs a new Brainbow technology for astrocytes by expressing two or three fluorescent proteins simultaneously.

The vehicle and the vehicle system of the present invention are both efficient and safe.

L-proline can induce a large number of astrocytes to take up the plasmid vector and express green fluorescent protein, and L-proline solution in high-concentration, and does not cause brain tissue cell apoptosis.

Astrocytes not only provide structural and energy support to neurons but also play an essential role in the blood-brain barrier. They also participate in the development of neurons and the brain's inflammatory response. Recently, the role of astrocytes in neural circuits has also attracted more and more attention. Corresponding with their various functions, the astrocytes may differ greatly in morphology and structure in different brain regions and even the same region. In the present invention, the glycine transfection system is used to express two or three plasmid vectors simultaneously. The efficient co-transfection of multiple plasmid vectors, provides a powerful tool for manipulating the expression of multiple genes in astrocytes simultaneously.

We also found that the uptake of different plasmid vectors by different astrocytes makes the expression of various fluorescent proteins very different. Using this result, we successfully constructed a new Brainbow technology for astrocytes by expressing two or three fluorescent proteins simultaneously. Compared with the previous Brainbow strategy, there is no need to construct a transgenic mouse or virus vector with a specific promoter, which significantly reduces the economic and time cost of multicolor labeling astrocytes. Plasma cell-neuron and astrocyte interactions provide a convenient and straightforward visualization tool.

Amino acid, is a group of organic molecules that consist of a basic amino group (—NH₂), an acidic carboxyl group (—COOH), and an organic R group (or side chain) that is unique to each amino acid. Some of the amino acids, when linked together with other amino acids, form a protein. Essential amino acids cannot be made by the body, and as a result, they must come from food. The 9 essential amino acids are: histidine, isoleucine, leucine, lysine, methionine, phenylalanine, threonine, tryptophan, and valine. Nonessential amino acids mean that our bodies produce an amino acid, even if we do not get it from the food we eat. Nonessential amino acids include: alanine, arginine, asparagine, aspartic acid, cysteine, glutamic acid, glutamine, glycine, proline, serine, and tyrosine.

The brain is an organ with a very complex structure and function. Each of these components has its unique function. The complex and precise coordination of interactions between different parts of the brain controls many different behaviors, such as cognition, feeling, movement, and emotions. Even at the macroscopically similar parts, there are considerable differences in the composition of cell types, interactions between cells, and molecular expression at the microscopic level. For a long time, astrocytes are generally considered to be a type of cells with similar morphology and function. However, in recent years, research on astrocyte diversity has attracted much attention.

Tree shrews are mainly distributed in South Asia, Southeast Asia, and Southwest China. Due to their small adult size, high brain-to-body mass ratio, short breeding cycle, simple breeding, and low cost, they are emerging experimental animals in the visual, Hepatitis virus infection and neurological diseases and have been extensively studied.

Microglia are innate immune cells present in the brain. There is increasing evidence that activated microglia are a chronic source of multiple neurotoxic factors, including TNFα, nitric oxide, Interleukin-1β, and reactive oxygen species(ROS). Iba1 (ionized calcium-binding adaptor molecule-1) protein is a microglial-specific marker protein. When brain tissue is damaged, microglia proliferate, and a series of morphological changes occur. Thus, the observation of microglial changes by immunofluorescence staining of Iba1 protein is often used to evaluate the inflammation or damage of brain tissues.

sgRNA can guide the endonuclease (Cas9 protein) to cut the target gene sequence accurately, so it has the potential to treat central nervous system diseases. Despite its vast application potential, effective and low toxicity delivery of genes related to the CRISPR/Cas9 system to the central nervous system is still challenging to achieve. At present, gene editing in the brain of adult mice is mainly achieved by viral vectors delivering sgRNA to target genes, but this method requires the construction of transgenic mice that constitutively express the Cas9 protein. Viral vectors are difficult to load the large open reading frame sequence (ORF) of Cas9 protein due to the size limitation of virus particles, which significantly limits the application of the CRISPR/Cas9 system in the central nervous system. Meanwhile, due to the inherent immunogenicity of viral vectors and the long-term expression of CRISPR-Cas9 and sgRNA, it is easy to cause off-target effects and thus induce potential carcinogenic risks. Therefore, in the central nervous system, by constructing viral vectors or transgenic mice, gene knock-down using the CRISPR-Cas9 system remains challenging.

Transposon. Non-viral vector nucleic acid delivery has great potential in genetic research and treatment due to its convenient and straightforward preparation method and high biological safety. Standard plasmid DNA delivered by non-viral vectors often cannot integrate the host genome, so these gene vectors are only transiently expressed in cells. However, the treatment of some hereditary or chronic diseases requires long-lasting gene expression. One of the methods to achieve long-term or even permanent stable expression of foreign genes is to use the transposable subsystem, which is a genetic element that can be transferred between the vector and the host genome or within the genome. Generally, transposases recognize specific inverted terminal repeat (IR) at both ends of the transposon and cut the transposon elements from their original positions to reintegrate them to other positions. Using these characteristics of the transposon, inserting the target gene between the terminal repeat sequences IRs at both ends of the transposon can integrate the foreign gene into the host's genome, and realize prolonged or even permanent expression of the foreign gene.

### BRIEF DESCRIPTION OF THE DRAWINGS

FIG. 1 shows the schematic diagram of the administration methods of delivery of plasmid vectors mediated by amino acids.

FIG. 2 shows the glycine solution promotes cellular uptake of plasmid vectors in the midbrain.

FIG. 3 shows the glycine transfection strategy specifically promotes astrocyte uptake of plasmid vectors.

FIG. 4 shows the validation of cell specificity of delivery mediated by glycine transfection strategy through transgenic mice (Aldh111-eGFP X Ai9).

FIG. 5 shows delivery efficiency and tissue damage of different concentrations of glycine transfection strategy.

FIG. 6 shows changes in microglia by immunofluorescence staining.

FIG. 7 shows that cellular uptake efficiency of plasmid vectors using a glycine transfection strategy is dependent on glycine concentration.

FIG. 8 shows the glycine transfection strategy promotes the uptake of plasmid vectors by astrocytes in different brain regions.

FIG. 9 shows the glycine transfection strategy promotes cellular uptake of plasmid vectors in the cerebellar cortex.

FIG. 10 shows the application of glycine transfection strategy in tree shrews.

FIG. 11 shows amino acids promote uptake of the plasmid vector by astrocytes.

FIG. 12 shows that the L-proline transfection system promotes plasmid uptake by astrocytes in different brain regions.

FIG. 13 shows the evaluation of the safety of the L-proline vehicle system on the central nervous system.

FIG. 14 shows GlyT1 selective inhibitor bitopertin increases the efficiency of uptake of plasmid vectors by glycine transfection system for 3-4 times.

FIG. 15 shows the glycine transfection strategy promotes the simultaneous delivery of two plasmid vectors.

FIG. 16 shows the simultaneous delivery of three plasmid vectors using glycine transfection strategy.

FIG. 17 shows different quantification of the color of astrocyte cell bodies.

FIG. 18 shows distinctive “territory” of astrocytes visualized by the multi-color strategy.

FIG. 19 shows the L-proline transfection system promotes efficient co-transfection using dual plasmid vectors and three plasmid vectors.

FIG. 20 shows the construction of gene targeting vectors for knockout of the eYFP gene.

FIG. 21 shows the expression of large plasmid vectors in astrocytes using a glycine transfection strategy.

FIG. 22 shows the schematic diagram of knocking out the eYFP gene of Ai3 transgenic mice by the CRISPR-Cas9 system.

FIG. 23 shows efficient eYFP gene knockout in astrocytes using glycine transfection strategy.

FIG. 24 shows validation of the knockout of eYFP gene by sequencing analysis.

FIG. 25 shows the schematic of integrating exogenous genes into the genome by the piggyBac (PB) transposon system.

FIG. 26 shows integrating eGFP genes into the genome of astrocytes by the piggyBac (PB) transposon system.

FIG. 27 shows PiggyBac transposon-mediated long-term eGFP expression in astrocytes of adult mouse brain.

Description of Particular Embodiments of the Invention

The descriptions of particular embodiments and examples are provided by way of illustration and not by way of limitation. Those skilled in the art will readily recognize a variety of noncritical parameters that could be changed or modified to yield essentially similar results.

### EXAMPLES

### Example 1. Plasmid Delivery of Glycine-Based Transfection System in Mouse Midbrain

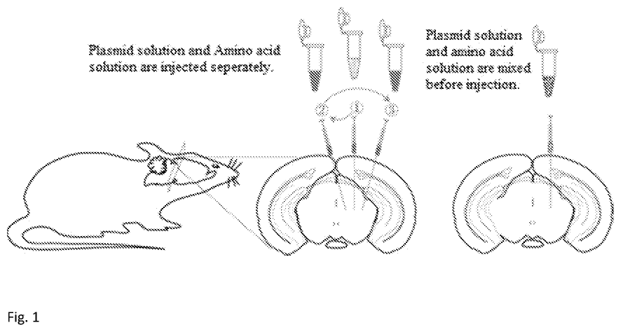

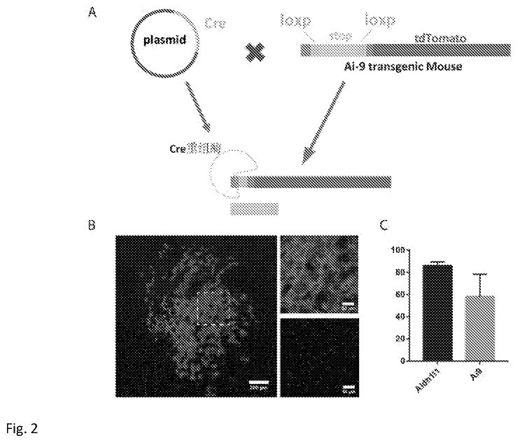

As shown in FIG. 1, a solution of the plasmid (pBT140) expressing Cre recombinant enzyme (1.5 μg/μL) and a glycine solution (300 mM) are injected into midbrain of Ai9 mice comprising the Cre-LoxP system. Cre recombinase will delete the termination sequence between LoxP sequences, thereby eliminating the effect of termination sequences on the transcriptional expression of the fluorescent protein tdTomato (FIG. 2A).

After 5 days, a large number of cells around the injection site were observed to express tdTomato in the sections. This result demonstrates that the transfection system composed of a glycine solution can effectively promote the uptake of plasmids by mesencephalic cells, and no visible tissue necrosis suggests that the transfection system has low toxicity (FIG. 2B). Also, a comparative analysis of the structure and morphology of fluorescent protein-expressing cells under a high magnification microscope revealed that all cells successfully transfected under this transfection system were astrocytes. The Aldh111-eGFP transgenic mouse is reporter line that specifically expresses a fluorescent protein (GFP) in astrocytes. By comparing astrocytes in Aldh111-eGFP mice and expressed in glycine transfection systems in Ai9 mice, the glycine transfection system is in the densely expressed area. The density of expressing cells reaches 67.7% (FIG. 2C).

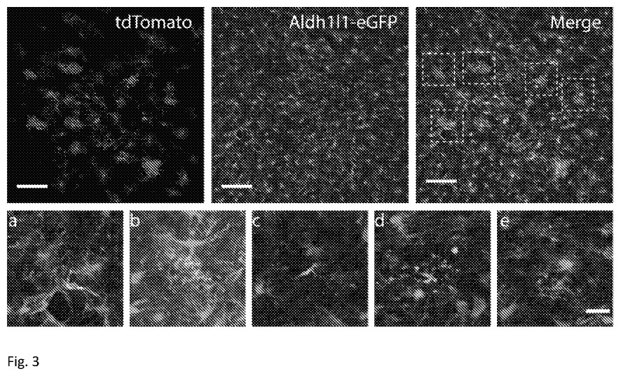

Fluorescent protein tdTomato is expressed in astrocyte-specifically labeled Aldh111-eGFP transgenic mice (FIG. 3). Through laser confocal microscope imaging analysis, it was found that all cells expressing tdTomato were astrocytes labeled in Aldh111-eGFP mice, indicating that the glycine solution specifically promotes astrocytes' uptake of the plasmid.

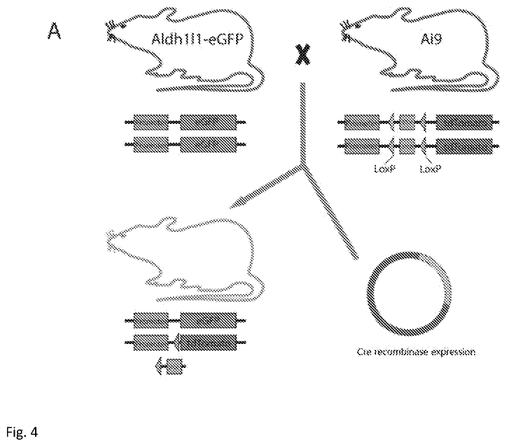

As shown in FIG. 4A, a hybrid mouse of Aldh111-eGFP mouse and Ai9 mouse (Aldh111-eGFP X Ai9) was constructed, which both specifically expresses eGFP in astrocytes, and expresses tdTomato by Cre recombinase. Cre recombinase is able to cleave LoxP sites even with extremely small or short-term expression, so it can continuously express the fluorescent protein tdToamto, thereby eliminating the problem that some cells express weakly and cannot be accurately observed. A glycine transfection system was injected into Aldh111-eGFP×Ai9 hybrid mice, and the cells expressing tdTomato fluorescent protein were still all astrocytes, which once again proved that this transfection method has cell specificity (FIG. 4B).

### Example 2. Evaluation of Damage of Glycine Vehicle System on the Central Nervous System

In order to evaluate whether the glycine transfection system would cause local or large-scale tissue damage to the brain tissue, we used transfection systems with different glycine concentrations and observed the sections 5 days after injection. Nucleus damage is an important mechanism for evaluating cytotoxicity. When a large number of cells die, and tissue necrosis, the nucleus of the cells will appear condensation, chromatin condensation and deformation, and nuclear fragmentation and dissolution.

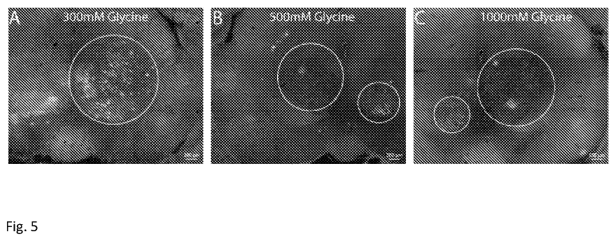

As shown in FIG. 5, the glycine transfection system of 300 mM induced the expression of a large number of astrocytes. The cell morphology was normal, and no visible tissue necrosis was observed. When using a higher concentration of the transfection system (500 mM and 1000 mM), the nucleus of a large number of cells around the injection site is condensed, and tissue damage is severe. At the same time, few astrocytes were expressed, and only a few were scattered around the damaged tissue. The cause of these damages may be the ultra-high concentration of glycine in local tissues on the one hand or may be caused by the osmotic pressure of the glycine transfection system exceeding 300 mM under normal physiological condition. By comparison, under the 300 mM glycine transfection system, the cell transfection efficiency is high, and the damage to the brain tissue is not obvious, indicating that the glycine transfection system is both efficient and safe.

In order to evaluate whether the glycine vehicle system will result in obvious inflammatory responses and tissue damage, we observe the change of microglia. Microglia are innate immune cells in the brain. More and more evidence shows that activated microglia are a chronic source of multiple neurotoxic factors, including TNFα, nitric oxide, Interleukin-1β, and reactive oxygen species (ROS).

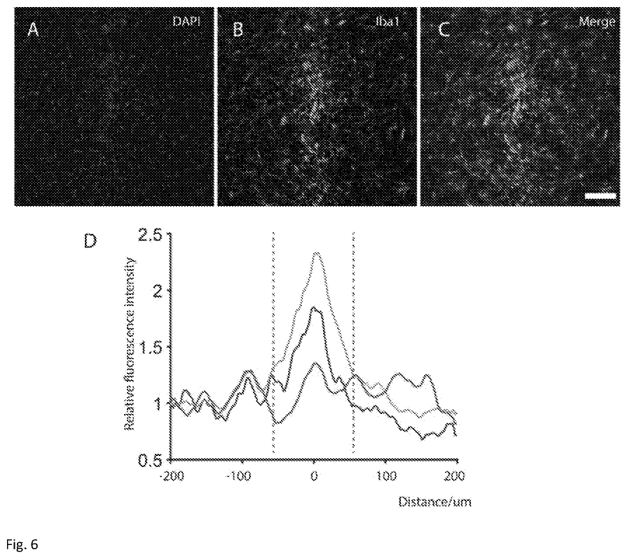

To evaluate whether the glycine transfection system would activate microglia, we used microglial-specific marker protein Iba1 for fluorescent immunostaining. As shown in FIGS. 6A, 6B and 6C, there are microglial activation and cell proliferation in a limited area (about 100 um) around the injection path, while in the area far away the injection path, although there are also a large number of astrocytes, microglia cells were not significantly activated (FIG. 6D), indicating that the mechanical damage caused by the injection electrode in brain tissue caused a certain degree of the inflammatory response, which in turn activated microglia. In contrast, the glycine transfection system did not cause significant inflammatory responses and tissue damage.

### Example 3. Glycine Concentration-Dependent Uptake of Plasmid by Astrocytes

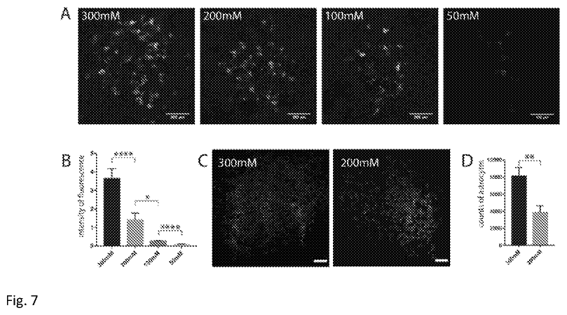

Glycine transfection systems of 300 mM, 200 mM, 100 mM, and 50 mM in an equal volume are injected into the mouse's midbrain tissue. After 5 days, the expression of the fluorescent protein was analyzed by laser confocal microscope and ImageJ software. As shown in FIGS. 7A and 7B, as the concentration of glycine decreases, the uptake efficiency of astrocytes on the plasmid vector significantly decreases. When using 50 mM concentration of glycine, only a few astrocytes take up and express green fluorescent protein (EGFP).

In addition, the plasmid vector pBT140 (expressing Cre recombinase) was injected into the midbrain of Ai9 transgenic mice, and astrocytes expressing the fluorescent protein tdTomato were counted by ImageJ software. As shown in FIGS. 7C and 7D, when glycine transfection system of 300 mM is used, the average number of astrocytes expressed is up to 8,000, and when the system of 200 mM is used, the number of cells is significantly reduced. These results indicate that changes of glycine concentration have a significant effect on the efficiency of astrocytes uptake of plasmids, and as the concentration of glycine decreases, the uptake efficiency decreases significantly.

### Example 4. Glycine Transfection System Promotes Plasmid Uptake by Astrocytes in Different Brain Regions

Because of huge differences in brain tissue function and structure, it is necessary to explore the effect of the glycine transfection system on promoting the efficiency of plasmid uptake by astrocytes in different brain regions.

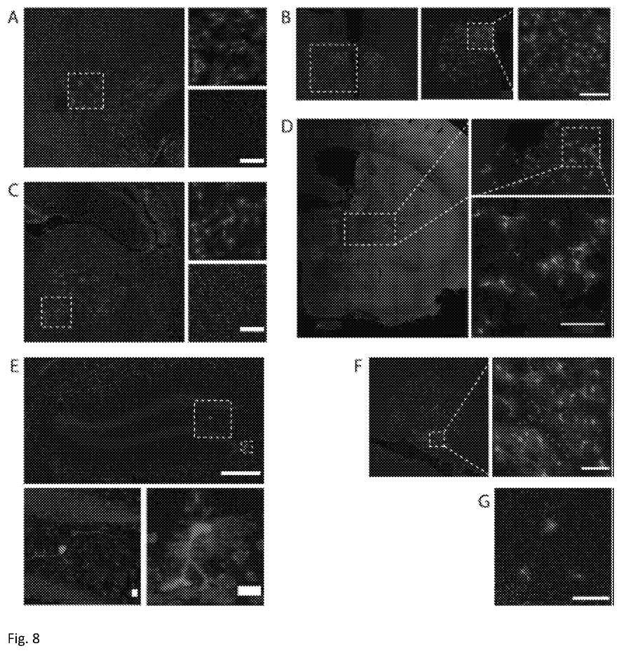

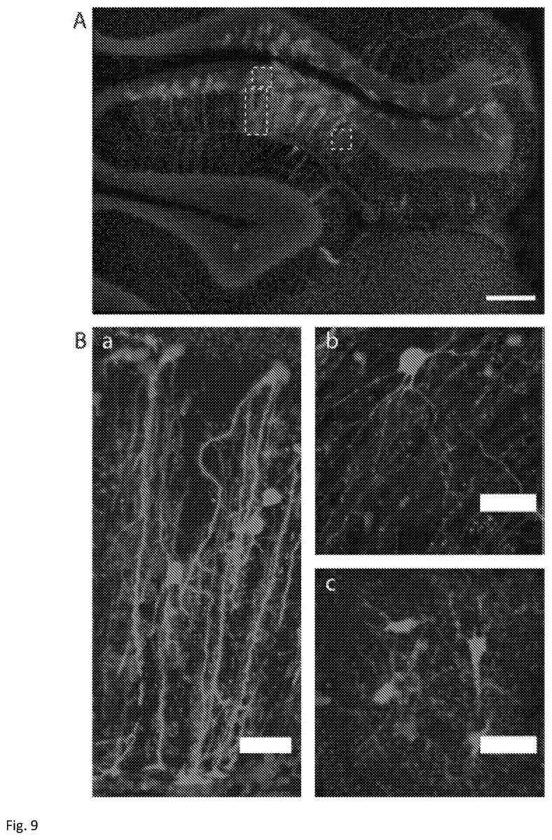

We tried to use glycine to promote the uptake of the plasmid (pBT140, which expresses Cre recombinase) in different brain regions of adult Ai9 mice. These brain regions include hypothalamus (FIG. 8A), thalamus (FIG. 8B), preoptic area of hypothalamus (FIG. 8C), globus pallidus lateralis of striatum (FIG. 8D), hippocampus (FIG. 8E), brain stem (FIG. 8F), cerebral cortex (FIG. 8G), and cerebellar cortex (FIG. 9). The results show that tdTomato fluorescent protein is expressed in a large number of astrocytes in the hypothalamus, thalamus, globus pallidus lateralis and brain stem, while only a few cells in the hippocampus and cortex express the fluorescent protein tdTomato. At the same time, neurons are present in cells expressed in the hippocampus. Interestingly, in the cerebellum, the glycine transfection system can efficiently promote plasmid uptake (FIG. 9A), and there are multiple cell types (FIG. 9B), including Bergman glial cells surrounding Purkinje cells, velate astrocytes surrounding granular cells, and basket/stellate intermediate neurons located in the molecular layer of the cerebellar cortex.

### Example 5. Use of Glycine Transfection System in Other Species

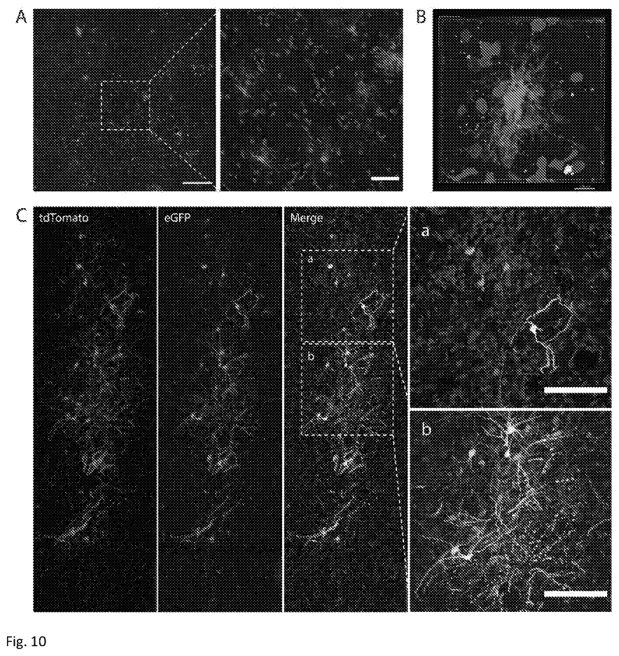

Chinese tree shrews (Tupaia belangeri chinensis) are selected as the experimental objects. Glycine transfection system of 300 mM is injected into the midbrain of the tree shrew in order to deliver a plasmid vector expressing tdTomato fluorescent protein. As shown in FIG. 10A and FIG. 10B, many astrocytes in the midbrain of tree shrews take up and express the plasmid vectors. Interestingly, when the glycine transfection system (two fluorescent protein vectors:eGFP and tdToamto) was injected into the striatum of tree shrews, it was found that many neurons expressed two fluorescent proteins (FIG. 10C). These results indicate that the use of the glycine transfection system can promote the uptake of plasmid vectors by astrocytes of other species.

### Example 6. Other Amino Acids Promote Uptake of Plasmid Vectors by Astrocytes

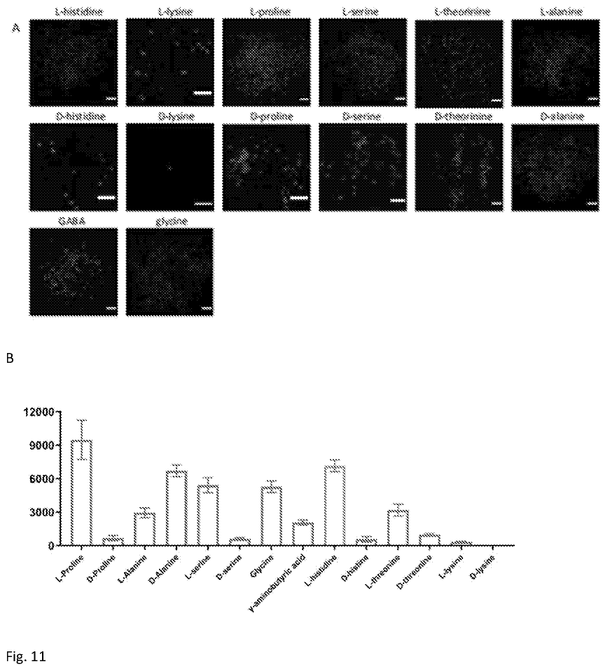

Other amino acids, including serine, alanine, histidine, γ-aminobutyric acid, proline, lysine, and threonine, are tested. It was found that these amino acids can efficiently promote astrocytes to take up plasmid vectors. From the analysis of the transfection efficiency of various amino acids, it was seen that, except for alanine, the efficiency of L-type amino acids is much higher than that of D-type amino acids (please see FIG. 11).

Histidine, lysine, and arginine, which are also positively charged, are significant differences in delivery efficiency, and gene delivery efficiency of threonine, serine lysine, etc. also has vast differences. These results show that the delivery efficiency of amino acid solution to gene carrier may have nothing to do with its physical and chemical properties, but may be related to the biological function of amino acids in the brain (Scalebar=200 μm).

### Example 7. L-Proline Transfection System Promotes Plasmid Uptake by Astrocytes in Different Brain Regions

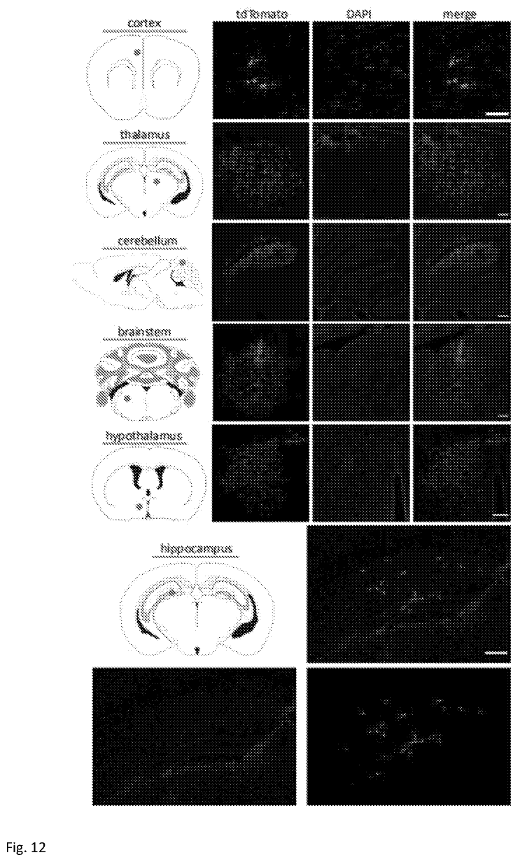

L-proline transfection system is used to promote the uptake of the plasmid (pBT140, which expresses Cre recombinase) in different brain regions of adult Ai9 mice. These brain regions include hypothalamus, thalamus, hippocampus, brain stem, cerebral cortex, and cerebellum. The results show that tdTomato fluorescent protein is expressed in a large number of astrocytes in the hypothalamus, thalamus, and brain stem, while only a few cells in the hippocampus and cortex express the fluorescent protein tdTomato. Interestingly, in the cerebellum, the L-proline transfection system can efficiently promote plasmid uptake (FIG. 12), and there are multiple cell types, including Bergman glial cells surrounding Purkinje cells, velate astrocytes surrounding granular cells, and basket/stellate intermediate neurons located in the molecular layer of the cerebellar cortex (FIG. 12).

### Example 8. Evaluation of Damage of L-Proline Vehicle System on Central Nervous System

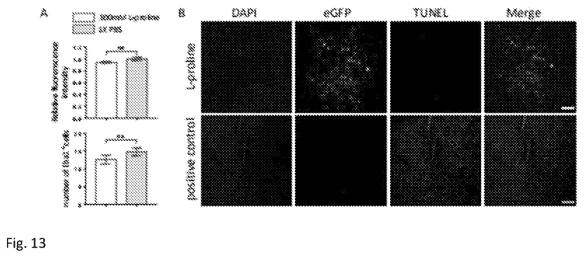

In order to evaluate whether a high concentration of L-proline solution induces the uptake of plasmid vector by astrocytes could cause visible central nervous system damage, microglial-specific marker protein Iba1 is used for fluorescent immunostaining. FIG. 13 shows that compared to the 1×PBS control group, the fluorescence intensity of Iba1 protein immunofluorescence staining and Iba1-labeled microglia in the test group did not increase significantly, indicating that L-proline solution of 300 mM did not cause significant tissue damage.

During apoptosis, genomic chromosomes are randomly cut by nucleases, and a large number of sticky 3′-OH ends are produced. Under the action of deoxyribonucleotide terminal transferase (TdT), fluorescent molecules can be labeled to 3′-terminus of DNA fragments, while normal cells have almost no 3′-OH terminus due to DNA breakage, so TUNEL method is a common method for detecting apoptosis. In order to investigate whether high-concentration L-proline solution (300 mM) can cause apoptosis of brain tissue cells, TUNEL staining was used to detect apoptosis in brain slices of proline-injected areas. It was found that L-proline can induce a large number of astrocytes to take up the plasmid vector and express green fluorescent protein, but almost no TUNEL fluorescent-labeled cells were found, indicating that high concentration of L-proline solution did not cause apoptosis in brain tissue cells (FIG. 13).

### Example 9. GlyT1 Selective Inhibitor Bitopertin Increases the Efficiency of Uptake of Plasmid Vectors by Glycine Transfection System for 3-4 Times

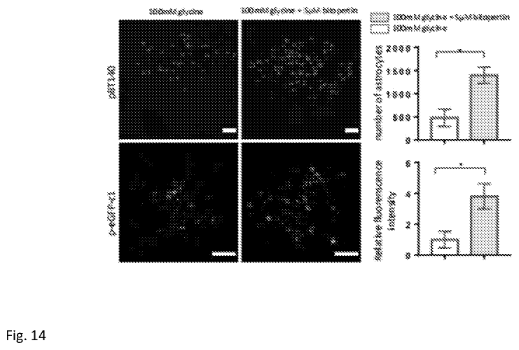

Astrocytes GlyT1 plays a vital role in the clearance of glycine in the extracellular space of brain tissue. Therefore, we used a selective inhibitor of GlyT1, bitopertin, to inhibit the astrocytes' clearance of glycine. It was found that the efficiency of uptake of plasmid vectors by cells was increased by 3-4 times, probably due to the inhibition of astrocytes through glycine (FIG. 14). The removal of extracellular glycine by the glycine transporter prolongs the action time of the high-concentration glycine solution, thereby improving the uptake of plasmid vectors by astrocytes.

### Example 10. Glycine Transfection System Promotes Efficient Co-Transfection of Dual Plasmid Vectors

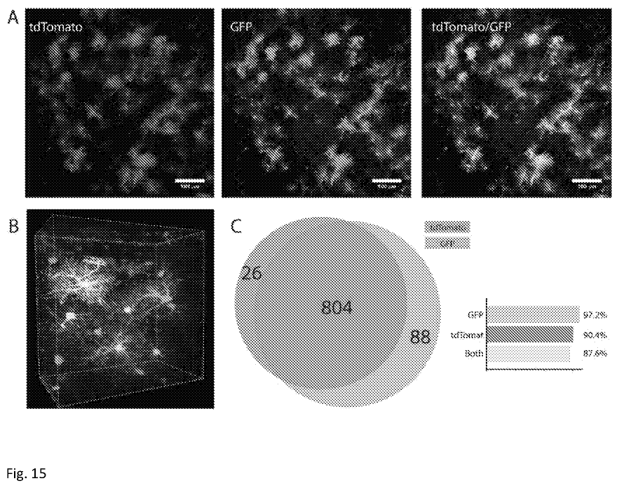

The plasmid vectors of green fluorescent protein and tdTomato fluorescent protein were selected as markers. As shown in FIG. 15A, two plasmid vectors were injected into the midbrain simultaneously. After 5 days, the brain tissue was taken through perfusing. Observation of brain sections by fluorescence microscopy revealed that both fluorescent proteins were highly expressed. Each astrocyte can be given a unique color mark. Therefore, the glycine transfection system not only achieves efficient transfection of two plasmids but also develops a very convenient and simple astrocyte Brainbow technology. As can be seen from the three-dimensional display of FIG. 15B, because each astrocyte has a different color from the adjacent cells, we can clearly distinguish the “domain” range of each astrocyte. In order to examine the co-transfection efficiency of the two plasmids, we counted a total of 918 astrocytes expressing fluorescent proteins (FIG. 15C). The statistical results show that the two plasmids have a co-transfection efficiency of up to 87.6%, indicating that the glycine transfection system can simultaneously induce efficient co-transfection of two plasmid vectors.

### Example 11. Glycine Transfection System Promotes Efficient Co-Transfection of Three Plasmid Vectors

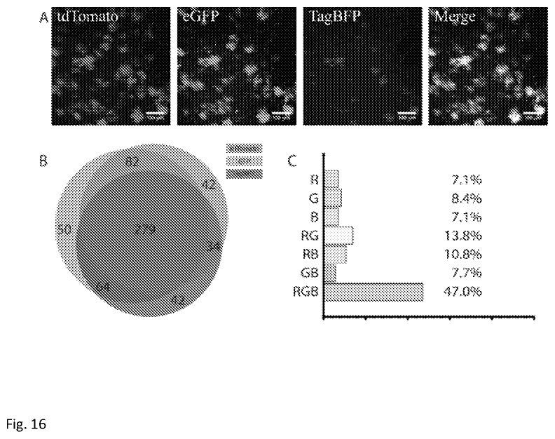

On the basis of eGFP and tdTomato plasmid vectors, TagBFP vector is added to test the efficient co-transfection of three plasmid vectors by glycine transfection system. The results showed that, all three plasmid vectors were highly expressed (FIG. 16A). At the same time, the maximum color combination of the Brainbow multicolor labeling strategy constructed using the dual plasmid vector is 255×255 in the RGB display mode, and after the BFP blue channel is introduced, the color mode was expanded to three-dimension information, reaching 255×255×255 in RGB display mode. The number of distinguishable colors is significantly expanded.

Confocal microscopy was used to capture astrocytes of different colors, and statistical analysis was performed on 593 astrocytes labeled on multiple brain slices (FIG. 16B). Nearly half of the labeled cells expressed three plasmid vectors (47%), and astrocytes expressing only two plasmid vectors accounted for 32.3% (FIG. 16C). These results show that the glycine transfection system can effectively promote the simultaneous uptake and expression of multiple plasmid vectors by astrocytes, and provide an effective gene expression tool for the simultaneous manipulation and study of multiple genes in adult mouse astrocytes. It also shows that the Brainbow strategy constructed using the glycine transfection system has excellent potential for multicolor labeling.

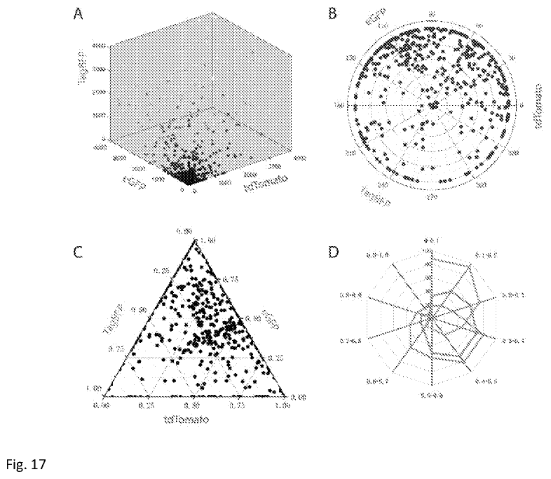

In order to further analyze the color distribution produced by the three-plasmid Brainbow strategy constructed using the glycine transfection system, a confocal microscope is used to capture, and imagJ software is used to obtain the color of each astrocyte cell body, which is presented in RGB mode. As shown in FIG. 17A, the specific values of each channel are directly plotted on the three-dimensional coordinate chart. It can be seen that in the entire three-dimensional coordinate, there are a certain number of cells distributed in different regions, but most of the cells are concentrated in darker regions. The results are similar to the expression of a two-plasmid vector. The three-dimensional coordinate rendering method using RGB mode can directly reflect the specific color of each astrocyte, but the color brightness of each cell depends on a variety of factors, such as imaging depth, cell morphology, and cell expression activity. Therefore, the RGB mode can be transformed into a two-dimensional HSB mode (hue-saturation-brightness mode) for display (as shown in FIG. 17B). The colors of different astrocytes are distributed in each area of the hue ring. Another way of presentation is through a ternary diagram with three axes, each axis representing a different color percentage, which can reflect the relative proportion of each fluorescent protein expression in different cells. As shown in FIG. 17C, the amount of TagBFP (blue channel) expressed by most cells (85.5%) is distributed below 50% of the highest fluorescence intensity (cells expressing the most TagBFP). As seen in FIG. 17D, the distribution of the three fluorescent proteins in the fluorescence intensity is also slightly different.

### Example 12. The Brainbow Strategy Constructed by Glycine Transfection System can Effectively Distinguish the “Territory” Occupied by Adjacent Astrocytes

Given the unique and complex three-dimensional structure of astrocytes, a clear distinction between different astrocytes can promote related research on interactions with neurons within the astrocyte “territory” and interactions between adjacent astrocytes.

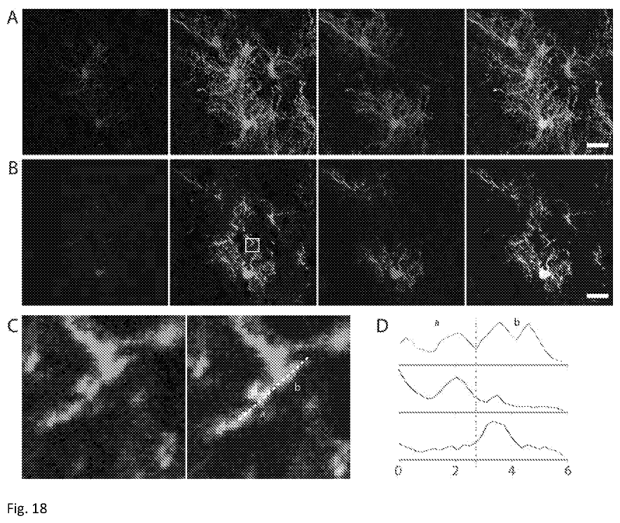

Glycine transfection system was used to construct a simple and convenient Brainbow technology for astrocytes. As shown in FIGS. 18A and 18B, in the local area, adjacent astrocytes were marked as different colors, the “territory” of each astrocyte is clearly discernible. Even in the extremely small space where adjacent astrocytes are in contact with each other, it is often difficult to accurately determine whether a microdomain belongs to two cells with a single-color marker. However, multi-color markers can clearly distinguish (FIGS. 18C and 18D).

### Example 13. L-Proline Transfection System Promotes Efficient Co-Transfection of Dual Plasmid Vectors and L-Proline Transfection System Promotes Efficient Co-Transfection of Three Plasmid Vectors

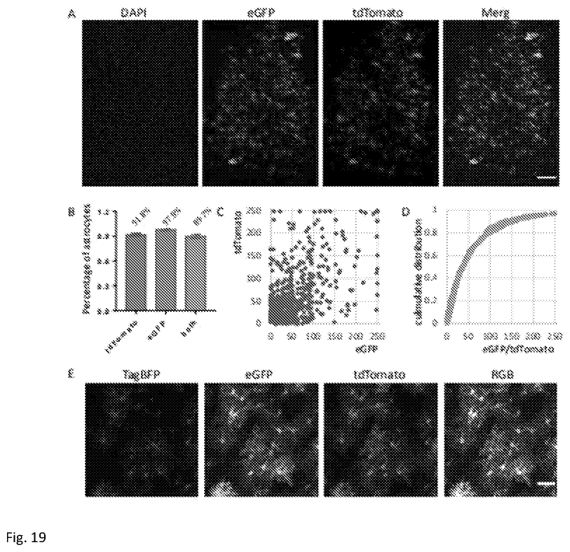

As shown in FIG. 19A, two plasmid vectors (p-eGFP-c1 and p-tdTomato-N1) was simultaneously expressed in the mouse midbrain with 300 mM of L-proline solution, the both fluorescent proteins are expressed in a large number of astrocytes, and meanwhile, astrocytes show different colors due to the difference in fluorescence expression (Scalebar=200 μm). As shown in FIG. 19B, among all astrocytes expressing fluorescent protein, 91.8% of cells express tdTomato and 97.9% of cells express eGFP, and the co-expression ratio reached 89.7%. FIGS. 19C-19D show distribution of cell color of different astrocytes. The two types of fluorescence intensity of the cell body are distributed throughout the brightness area, but mainly in darker areas. The number of cells with a cell body fluorescence intensity of RGB value less than 50 accounts for 60.9% (green) and 58.3% (red), respectively. FIG. 19E shows the simultaneous expression of three plasmid vectors (p-eGFP-c1, p-tdTomato-N1, and p-TagBFP-N at a concentration of 1 μg/μL respectively) in the mouse midbrain with 300 mM L-proline solution (Scalebar=50 μm).

### Example 14. Expression of Oversized Plasmid Vectors in Astrocytes

1. Constructing pX330-U6-sgRNA-CBh-hSpCas9-mCherry Plasmid

pX330-U6-sgRNA-CBh-hSpCas9-mCherry plasmid knocks out the EYFP gene of Ai3 mice and expresses the red fluorescent mCherry fluorescent protein.

The plasmids p-eGFP-c1, pBT140, pCAG-Pbase and PBCAG-eGFP were purchased from the Addgene platform. pX330-U6-Chimeric_BB-CBh-hSpCas9-mCherry was provided by Jiankui Zhou of Xingxu Huang′ lab.

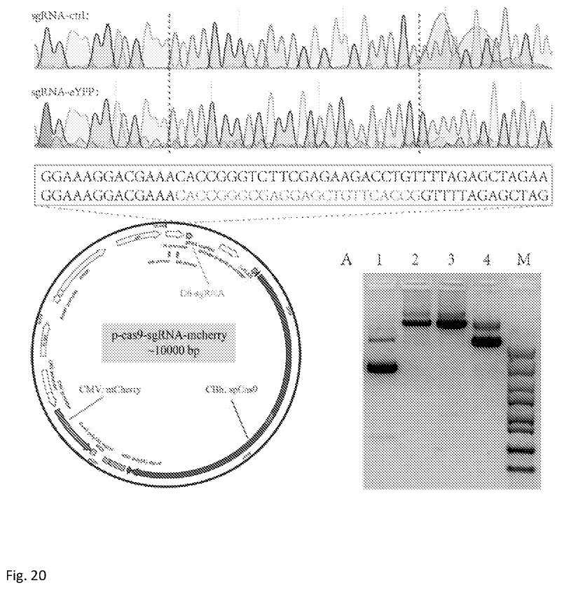

The pX330-U6-Chimeric_BB-CBh-hSpCas9-mCherry plasmid was used as a vector, and the sgRNA target binding sequence was inserted at the Bbs1 restriction site behind the U6 promoter. The target sequence of the sgRNA is EYFP sgRNA1-s: CAC CGG GCG AGG AGC TGT TCA CCG, EYFP sgRNA1-a: AAA CCG GTG AAC AGC TCC TCG CC. Firstly, the digestion of the vector plasmid is carried out using the digestion system (BbsI endonuclease 2 μl, vector plasmid 1-2 μg, adding digestion buffer and finally adding ddH₂O to 50 μl), and the reaction is performed at 37° C. for 30 min to 1 h. Takara miniBEST DNA Fragment Purification Kit ver4.0 kit was used to purify the digested product, and the digestion was detected by 1% agarose gel electrophoresis. The recovered digested fragment was ligated with the target sequence using T4 ligase. The enzyme ligation reaction system is: 100 ng of fragment, 1 μl of 5 mM target sequence hybrid double-stranded, 0.2-1 μl of T4 ligase enzyme, ligation buffer, adding ddH₂O to 10 μl), and reacts at constant temperature for 15-30 minutes. After bacterial transformation, single colonies are picked, and sequencing is conducted. At the same time, 500 μl of the bacterial solution is mixed with an equal volume of 50% sterile glycerol, and then select the corresponding bacterial solution for plasmid extraction according to the sequencing results. The constructed pX330-U6-sgRNA-CBh-hSpCas9-mCherry plasmid was shown in FIG. 20.

2. Expression of Oversized Plasmid Vectors in Astrocytes

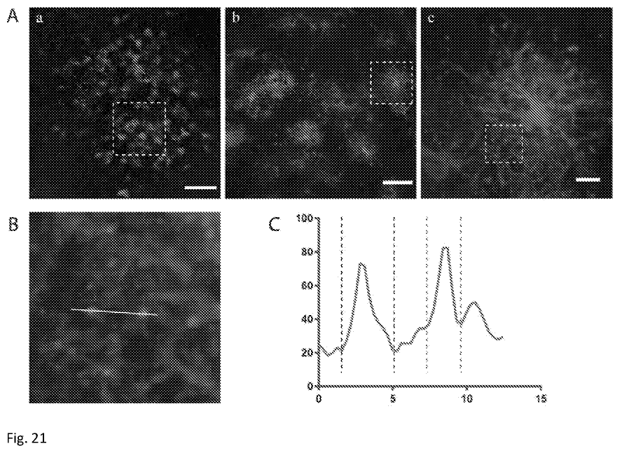

pX330-U6-sgRNA-CBh-hSpCas9-CMV-mCherry vector, which is approximately 10,000 base pairs in size, was chosen to express sgRNA through the U6 promoter, and SpCas9 protein through the SpB9 promoter, and express mCherry fluorescence through the CMV promoter. Glycine transfection system is used to deliver pX330-U6-sgRNA-CBh-hSpCas9-CMV-mCherry vector. As shown in FIG. 21A, a large number of astrocytes express fluorescent protein mCherry under a fluorescence confocal microscope, indicating that the vector can be efficiently taken up by astrocytes under the glycine transfection system. In addition, unlike other fluorescent proteins (eGFP, eYFP, tdTomato, TagBFP), the expressed mCherry fluorescent protein converge into small particles in astrocytes (FIG. 21B). Analysis using ImageJ software shows that these small particles have the diameter of about 1-3 μm.

### Example 15. Knocking Out the eYFP Gene of Ai3 Transgenic Mice Using the CRISPR-Cas9 System

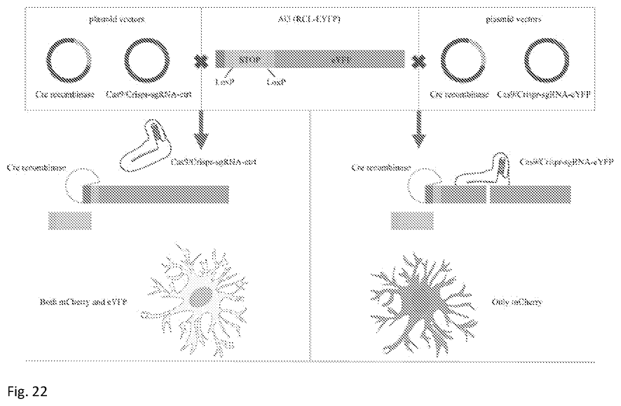

Ai3 mouse (RCL-EYFP) is selected as the reporter mouse, and the CRISPR/Cas9 system is used to target knock out the eYFP gene in its astrocyte genome. As shown in FIG. 22, two plasmid vectors (pBT140 vector expressing Cre recombinase, and pX330-U6-sgRNA-CBh-hSpCas9-mCherry vector expressing CRISPR/Cas9 system) are delivered using glycine transfection system, and when Cre recombinase vector is expressed in astrocytes, the termination sequence “STOP” in front of the eYFP gene sequence can be excised, thereby starting the expression of the eYFP gene, and when the CRISPR/Cas9 vector targeting the eYFP gene is simultaneously expressed in the cell, the eYFP gene is knocked out. So, even if the Cre recombinase exists, active eYFP fluorescent protein cannot been efficiently expressed.

Firstly, a suitable sgRNA that targets the eYFP gene was constructed. As shown in FIG. 20A, through 1% agarose gel electrophoresis, it can be seen that the digested plasmid vector produces a band of about 5 kb (Lane 1). Because in the vector pX330-U6-sgRNA-CBh-hSpCas9-mCherry, in addition to two BbsI restriction endonuclease sites in the sgRNA position, for inserting new sgRNA, in the mCherry fluorescent protein sequence, there is also a BbsI digestion site, which produces two fragments of similar size after digestion. Further, we confirmed by bacterial liquid sequencing (FIG. 20B) that the pX330-U6-sgRNA (eYFP)-CBh-hSpCas9-mCherry vector targeting the eYFP gene was successfully constructed.

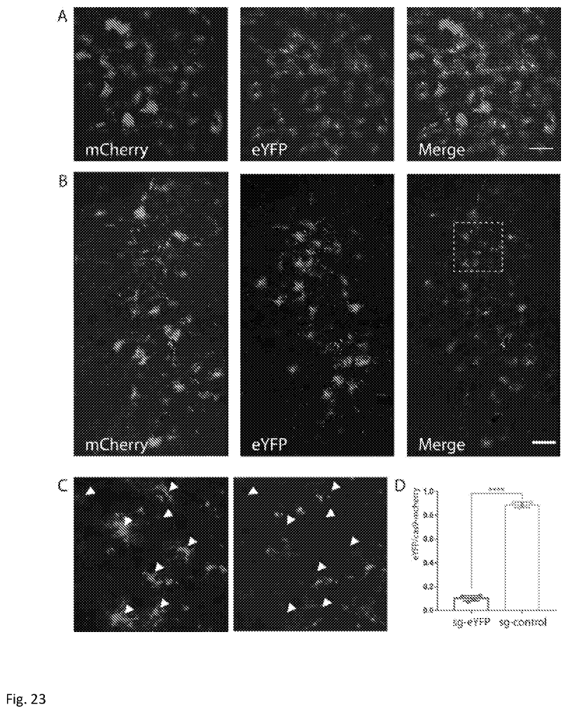

Next, in order to confirm whether the designed sgRNA-eYFP can effectively target and excise the eYFP gene in astrocytes from Ai3 transgenic mice. According to the design method (FIG. 22), two plasmid vectors pBT140 and pX330-U6-sgRNA (ctrl)-CBh-hSpCas9-mCherry were firstly injected into 8-week-old Ai3 transgenic mice. As shown in FIG. 23A, both eYFP and mCheryy fluorescent proteins were highly expressed, and most (average 88.7%) astrocytes expressing mCherry fluorescent protein also expressed eYFP fluorescent protein (FIG. 23D), indicating that the expression of CRISPR/Cas9 vector as a control group did not affect the expression of eYFP fluorescent protein. Two plasmid vectors pBT140 and pX330-U6-sgRNA (eYFP)-CBh-hSpCas9-mCherry were also injected simultaneously, as shown in FIGS. 23B and 23C, most (average 89.8%) astrocytes expressing mCherry did not express eYFP fluorescent protein, and further statistical analysis showed that sgRNA (eYFP) expression can effectively knock out the eYFP gene of Ai3 transgenic mouse astrocytes.

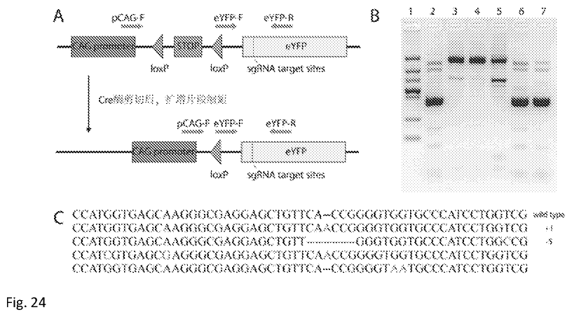

Therefore, in order to further verify the removal efficiency of CRISPR/Cas9, we sequenced the genome of the target location. As shown in FIG. 24A, we designed a pair of primers pCAG-F and eYFP-R to amplify the sequence containing the sgRNA target site by PCR. After Cre recombinase cleavage, the amplified fragments will be shortened. Through the optimization of PCR conditions, a cutoff of 300 bp was acquired for sequencing analysis (FIG. 24B). The sequencing results are shown in FIG. 24C. In astrocytes expressing Cre recombinase, insertion mutations, deletion mutations, and point mutations were detected. Point mutations have a limited effect on the structure of fluorescent proteins, which may be the reason why a small number (10.2%) of astrocytes expressing mCherry fluorescent protein can also see eYFP fluorescence.

### Example 16. Knocking-In the eGFP Gene in C57BL/6 Mice Using the piggyBac Transposon System

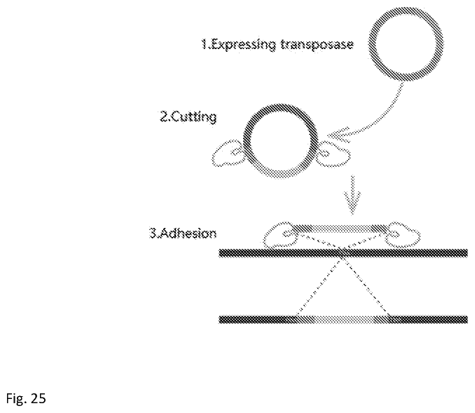

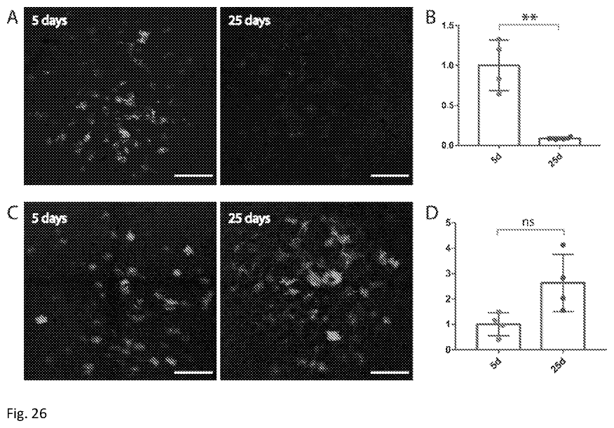

The piggyBac transposon system requires two plasmid vectors to express the transposase (pCAG-Pbase plasmid vector), and a transposon carrying a foreign gene with a terminal repeat sequence (PBCAG-eGFP plasmid vector) (FIG. 25). Firstly, a common plasmid vector (p-eGFP-C1) was injected. Since the common vector cannot be integrated into the cell's genome, the plasmid DNA can only be transiently expressed and is eventually degraded by the nuclease in the cell. As shown in FIG. 26A, after 25 days of expression, the intensity of green fluorescent protein (eGFP) decreased significantly, only about one-tenth of the fluorescence expression at 5 days (FIG. 26B). In contrast, when using the piggyBac transposon system, after 25 days of expression, compared with 5 days, the fluorescent protein expression was higher (FIG. 26C and FIG. 26D). The reason for the increase in fluorescence intensity is that through the transposon system, the eGFP gene is integrated into the genome, and the sustained and stable expression of eGFP increases the total amount of fluorescent proteins in the cell.

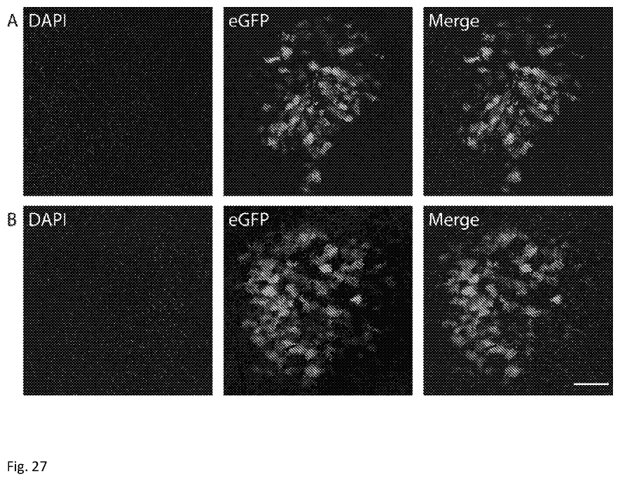

In order to further investigate the ability of the piggyBac transposon system to achieve long-term or even permanent expression of foreign genes, PB plasmid vector expression was observed for two months (FIG. 27A) and six months (FIG. 27B) respectively after injection. It was found that even up to half a year, eGFP expression remained stable and continued. These results fully demonstrate that the use of the glycine transfection system can induce the piggyBac transposon system to permanently express foreign genes in astrocytes of adult mice.

What is claimed is:

1. A vehicle system for delivery into living organisms, wherein the vehicle system comprises an amino acid and a nucleic acid.

2. The vehicle system of claim 1, wherein the nucleic acid is selected from oligonucleotide and plasmid.

3. The vehicle system of claim 1, wherein the vehicle system comprises an amino acid and one or more plasmids.

4. The vehicle system of claim 1, wherein the amino acid is selected from Glycine, GABA (γ-aminobutyric acid), Proline, Alanine, Serine, Histidine, and Threonine.

5. The vehicle system of claim 1, wherein the nucleic acid is dissolved in the solution of the amino acid before delivery, or the nucleic acid and the solution of the amino acid are delivered separately.

6. The vehicle system of claim 1, which further comprising a selective inhibitor of amino acid transporter.

7. The vehicle system of claim 1, wherein the nucleic acid is a CRISPR/Cas9 system.

8. The vehicle system of claim 1, wherein the nucleic acid is a transposon system.

9. The vehicle system of claim 1, wherein the concentration of the amino acid in the vehicle system is in the range of 50 mM-300 mM.

10. A composition for delivery of a nucleic acid into living organisms, comprising an amino acid, and a selective inhibitor of amino acid transporter.

11. The composition of claim 10, wherein the nucleic acid is selected from a short length single-stranded DNA, RNA, a CRISPR/Cas9 system and a transposon system.

12. The composition of claim 10, wherein the nucleic acid is one or more plasmids expressing one or more fluorescent proteins selected from RFP, YFP, GFP, CFP and BFP.

13. The composition of claim 10, wherein the amino acid is selected from Glycine, GABA (γ-aminobutyric acid), Proline, Alanine, Serine, Histidine, and Threonine.

14. The composition of claim 10, which is in the form of solution.

15. The composition of claim 10, wherein the concentration of the amino acid is in the range of 50 mM-300 mM.

16. Use of the vehicle system of claim 1 for delivery of a nucleic acid into living organisms, or use of a vehicle or the vehicle system of claim 1 in gene therapy for treating diseases.

17. The use of claim 16, wherein the nucleic acid is selected from a short length single-stranded DNA, RNA, for example, antisense RNA or siRNA, a CRISPR/Cas9 system, a transposon system.

18. The use of claim 16, wherein the nucleic acid is one or more plasmids expressing one or more fluorescent proteins selected from RFP, YFP, GFP, CFP and BFP.

19. The use of claim 16, wherein the amino acid is selected from Glycine, GABA (γ-aminobutyric acid), Proline, Alanine, Serine, Histidine, and Threonine.

20. The use of claim 16, wherein the vehicle system is in the form of solution.

21. The use of claim 16, wherein the vehicle directly delivers the nucleic acid into tissues or organs of living organisms, including but not limited to plant and animals, selected from invertebrates or vertebrates.

22. The use of claim 21, wherein the tissue or organ is the central nervous system, including the brain and spinal cord.

23. The use of claim 16, wherein the vehicle directly delivers the nucleic acid into astrocytes within midbrain, thalamus, hypothalamus, brainstem, cerebellum, globus pallidus lateralis, cerebral cortex and/or hippocampus.

24. The use of claim 16, wherein the vehicle directly delivers the nucleic acid into Bergman glial cells, velate astrocytes, and basket/stellate intermediate neuron within the cerebellum.

25. The use of claim 16, wherein the concentration of the amino acid is in the range of 50 mM-300 mM.

26. The use of claim 16, wherein the disease is a diseases of central nervous system.

27.-40. (canceled)
